(19) United States
(12) Patent Application Publication
(10) Pub. No.: US 2024/0055109 A1
(43) Pub. Date: Feb. 15, 2024

(54) INSPECTION ASSISTANCE DEVICE, INSPECTION ASSISTANCE METHOD, AND RECORDING MEDIUM

(71) Applicant: NEC Corporation, Minato-ku, Tokyo (JP)

(72) Inventors: Ayaka AMAKAWA, Tokyo (JP); Kenichi Kamijo, Tokyo (JP); Yatin Joshi, Tokyo (JP)

(73) Assignee: NEC Corporation, Minato-ku, Tokyo (JP)

(21) Appl. No.: 18/267,599

(22) PCT Filed: Mar. 25, 2021
(86) PCT No.: PCT/JP2021/012497

§ 371 Date: Jun. 15, 2023

(51) Int. Cl. G16H 40/20; G16H 30/40

(52) U.S. Cl. CPC G16H 40/20; G16H 30/40

(57) ABSTRACT

The present invention assists with genetic testing in which pathological image data is used. A classifying unit (11) uses pathological image data, which is the test subject of genetic testing, to classify the test object by tumor cell type. A selecting unit (13) selects, from among available pathologists who can be asked to assist, a pathologist candidate to be asked to assist with the genetic testing, on the basis of the tumor cell type. A presenting unit (14) displays the pathologist candidate.

### TECHNICAL FIELD

The present invention relates to an inspection assistance device, an inspection assistance method, and a recording medium, and particularly relates to an inspection assistance device, an inspection assistance method, and a recording medium that assist an inspection of tumor cells using a pathology specimen.

### BACKGROUND ART

In order to acquire a cell population to be subjected to a genetic test of somatic cells, a device in which a laser irradiation device is connected to a microscope is used. In one method, the sliced tissue is attached to a dedicated slide and stained, and then a laser is radiated along the contour of the test region while observing a section of the tissue with a microscope. As a result, the cell population in the test region can be separated from the tissue and collected. This is an example of a technique called die section (dissociation).

In a related technology, diagnosis and diagnosis assistance are performed using a digitized pathological image. For example, in the related technique described in PTL 1, a pathological tissue is automatically identified using a pathological image with a high magnification and a pathological image with a low magnification. This technique is an example of digital pathology.

PTL 2 describes that image processing for die section assistance is performed on pathological image data, and the processed pathological image data is transmitted to a terminal of a pathologist.

### CITATION LIST

### Patent Literature

PTL 1: JP 2010-203949 A

PTL 2: WO 2011/108551 A1

### SUMMARY OF INVENTION

### Technical Problem

For an accurate genetic test, it is required to use pathological image data including a test region having a high tumor cell content ratio. However, it is difficult for a pathologist to appropriately determine a test region having a high tumor cell content ratio. In such cases, an unskilled pathologist would like to be advised or assisted by a skilled pathologist, but the skilled pathologist is not always nearby.

The present invention has been made in view of the above problems, and an object thereof is to assist a genetic test using pathological image data.

### Solution to Problem

An inspection assistance device according to an aspect of the present invention includes a classification means configured to classify, using pathological image data as a test target of a genetic test, the test target by tumor cell type, a selection means configured to select one or more pathologist candidates who are requested for assistance for the genetic test from among pathologists who are capable of being requested for assistance based on a type of tumor cell related to the test target, and a presentation means configured to present the pathologist candidates.

An inspection assistance method according to an aspect of the present invention includes classifying, using pathological image data as a test target of a genetic test, the test target by tumor cell type, selecting one or more pathologist candidates who are requested for assistance for the genetic test from among pathologists who are capable of being requested for assistance based on a type of tumor cell related to the test target, and presenting the pathologist candidates.

A non-transitory storage medium according to an aspect of the present invention stores a program for causing a computer to execute classifying, using pathological image data as a test target of a genetic test, the test target by tumor cell type, selecting one or more pathologist candidates who are requested for assistance for the genetic test from among pathologists who are capable of being requested for assistance based on a type of tumor cell related to the test target, and presenting the pathologist candidates.

### Advantageous Effects of Invention

According to an aspect of the present invention, it is possible to assist a genetic test using pathological image data.

### BRIEF DESCRIPTION OF DRAWINGS

FIG. 1 is a diagram illustrating an example of transmission and reception of data in a system including a terminal of a pathologist and a server.

FIG. 2 is a diagram illustrating an example of image data of a pathology specimen.

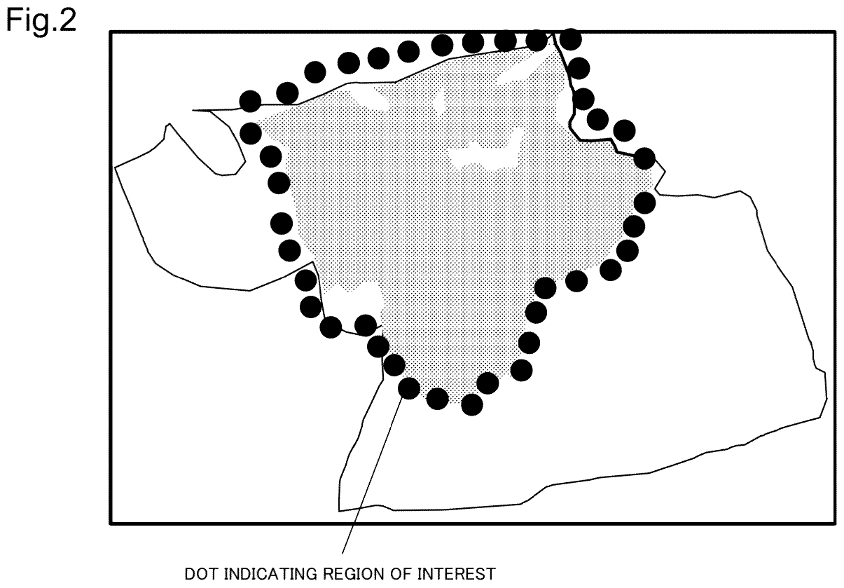

FIG. 3 is a block diagram illustrating a configuration of an inspection assistance device according to the first example embodiment.

FIG. 4 is a flowchart illustrating an operation of the inspection assistance device according to the first example embodiment.

FIG. 5 is a diagram illustrating a flow of data in a system including an inspection assistance device (cloud server) according to the second example embodiment.

FIG. 6 is a block diagram illustrating a configuration of the inspection assistance device according to the second example embodiment.

FIG. 7 is a flowchart illustrating an operation of the inspection assistance device according to the second example embodiment.

FIG. 8 is a diagram illustrating a flow of data in a system including an inspection assistance device (cloud server) according to the third example embodiment.

FIG. 9 is a block diagram illustrating a configuration of the inspection assistance device according to the third example embodiment.

FIG. 10 is a flowchart illustrating an operation of the inspection assistance device according to the third example embodiment.

FIG. 11 is a diagram illustrating an example of a hardware configuration of the inspection assistance device according to any one of the first to third example embodiments.

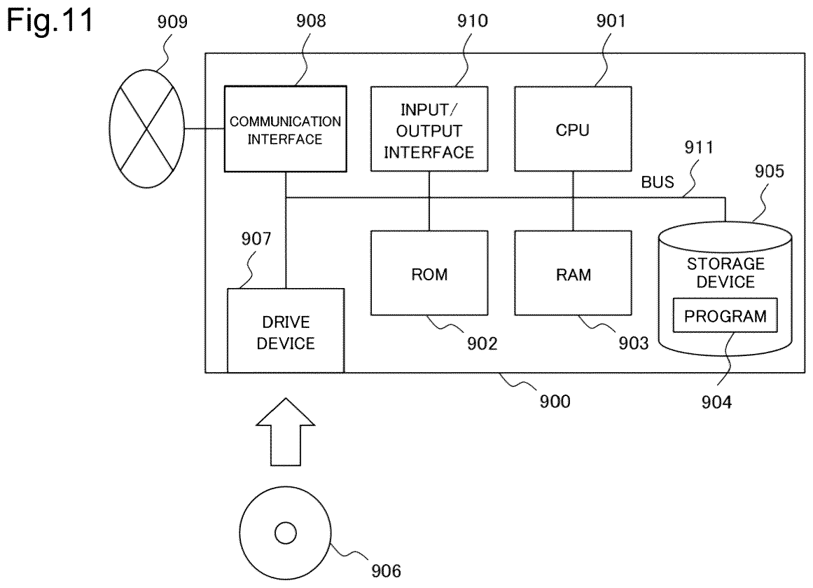

### EXAMPLE EMBODIMENT

Example embodiments of the present invention will be described with reference to the drawings.

### System 1

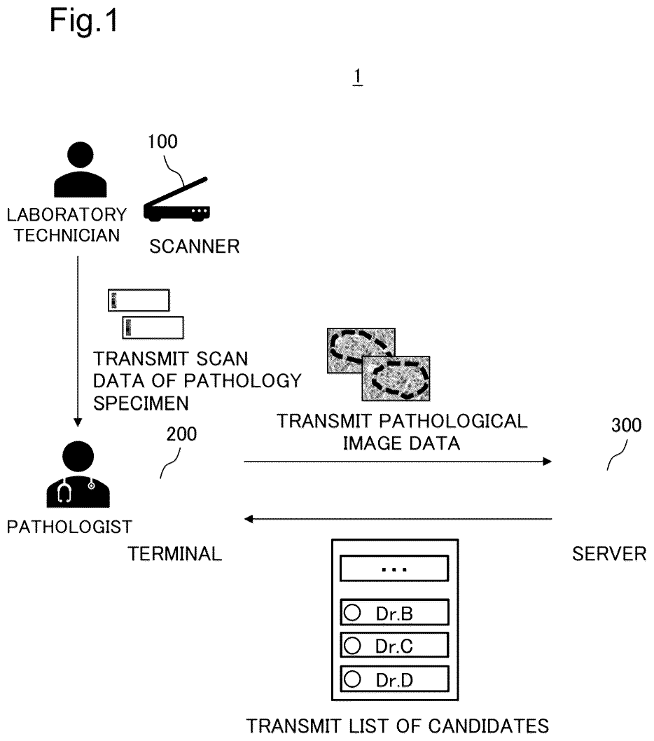

Referring to FIG. 1, an example of a configuration of a system 1 to which inspection assistance devices 10 to 30 according to the first to third example embodiments to be described later are applied will be described. FIG. 1 is a diagram schematically illustrating an example of a configuration of the system 1.

As illustrated in FIG. 1, the system 1 includes a scanner 100 of a laboratory technician, a terminal 200 of a pathologist, and a server 300.

The laboratory technician creates a pathology specimen of cellular tissue to be subjected to a genetic test. The laboratory technician creates scan data of the pathology specimen using the scanner 100. The laboratory technician transmits the created scan data of the pathology specimen from the scanner 100 to the terminal 200 of the pathologist.

By processing the scan data of the pathology specimen received by the terminal 200, the pathologist creates image data (hereinafter, referred to as pathological image data) of a pathology specimen to be tested. For example, the pathologist determines a region of interest where there are many cells in which the ratio of tumor cells is considered to be high based on the concentration of staining of the tissue.

The pathologist annotates the region of interest. For example, the pathologist may add a marking to a region of interest in which the ratio of the region of tumor cells is considered to be high on the image data of the pathology specimen using general image editing software. For example, the pathologist inputs dots or lines in such a way as to surround the region of interest on the image data of the pathology specimen using general image editing software operating on the terminal 200. Alternatively, the pathologist may acquire the pathology specimen itself created by the laboratory technician, and after drawing dots or lines on the pathology specimen with a magic pen or the like, may scan the pathology specimen using the scanner 100. The terminal 200 of the pathologist transmits the pathological image data created in this manner together with the request for assistance to the server 300. The case where the laboratory technician designates the region of interest is described. However, the region of interest does not necessarily need to be designated. In a case where the region of interest is not designated, in the following description, the “region of interest” is read as the entire image data of the pathology specimen.

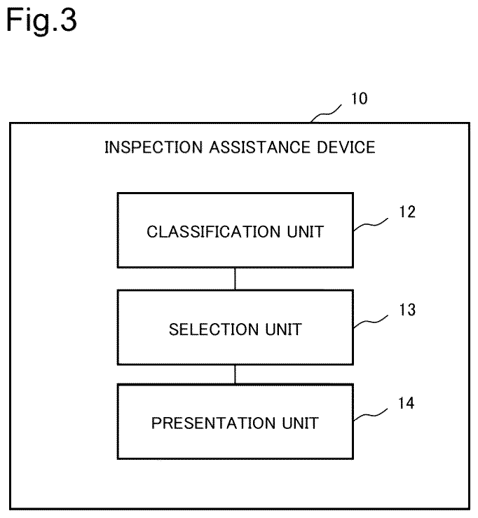

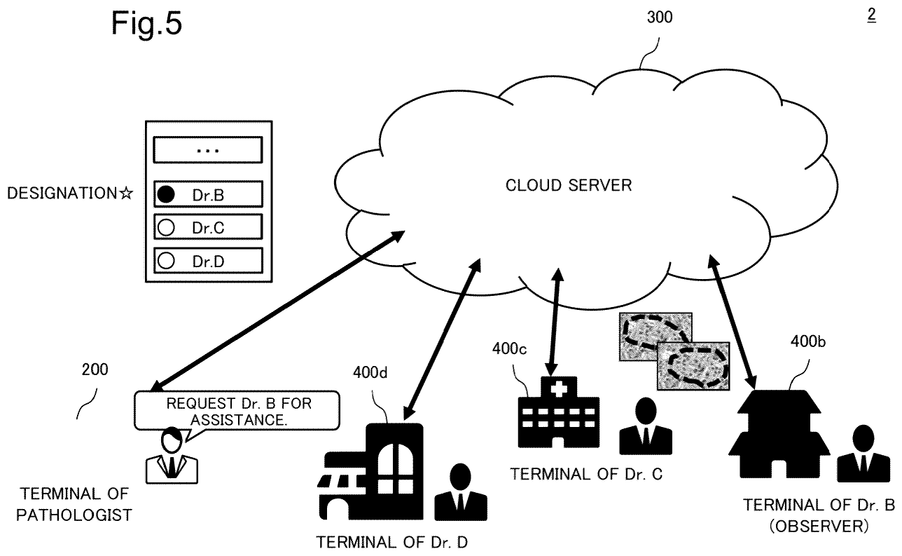

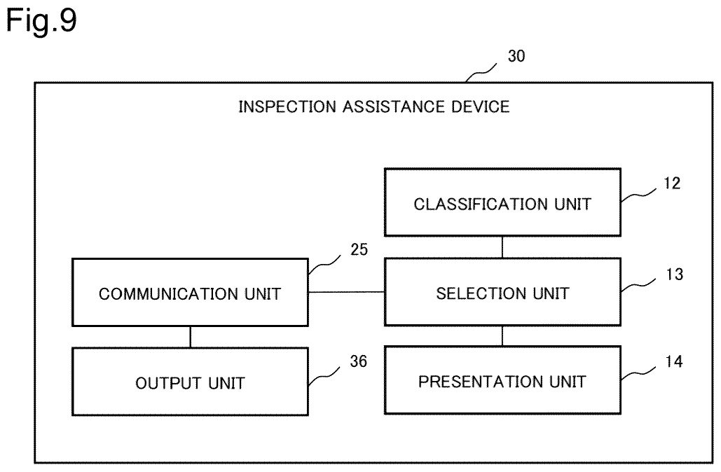

The server 300 includes any one of an inspection assistance device 10 (FIG. 3), an inspection assistance device 20 (FIG. 5), and an inspection assistance device 30 (FIG. 9) according to the first to third example embodiments to be described later. Hereinafter, any one of the inspection assistance devices 10 to 30 will be referred to as a “inspection assistance device 10 (20, 30)”.

The server 300 transmits information indicating the pathologist candidate who is requested for assistance to the terminal 200 of the pathologist. For example, the server 300 transmits the list of candidates to the terminal 200 of the pathologist.

The terminal 200 of the pathologist receives the list of candidates from the server 300. A detailed configuration and operation of the inspection assistance device 10 (20, 30) included in the server 300 will be described in the first to third example embodiments.

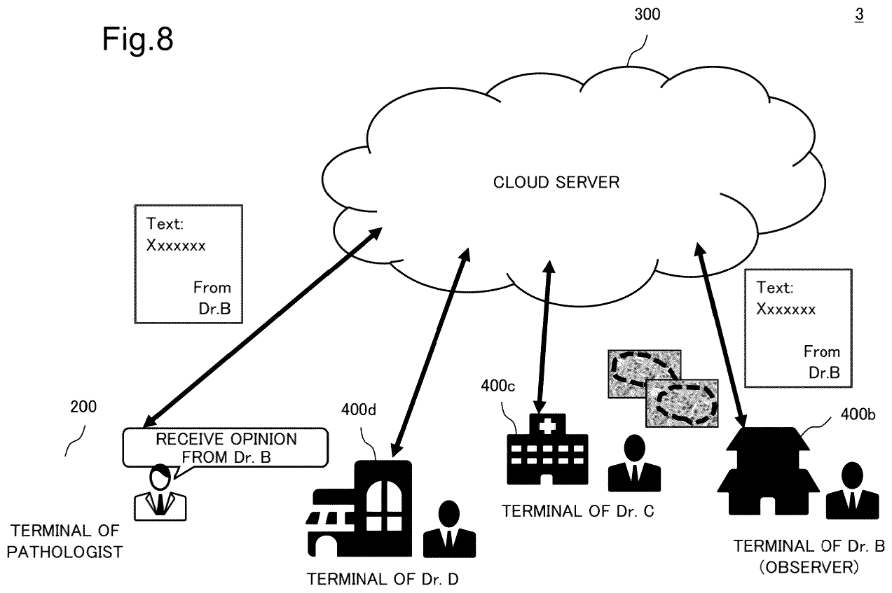

The configuration of the system 1 including the inspection assistance device 10 (20, 30) according to the first to third example embodiments is not limited to the example illustrated in FIG. 1. In the second example embodiment to be described later, another system 2 (FIG. 5) including the inspection assistance device 20 will be described. In the third example embodiment to be described later, another system 3 (FIG. 8) including the inspection assistance device 30 will be described.

### First Example Embodiment

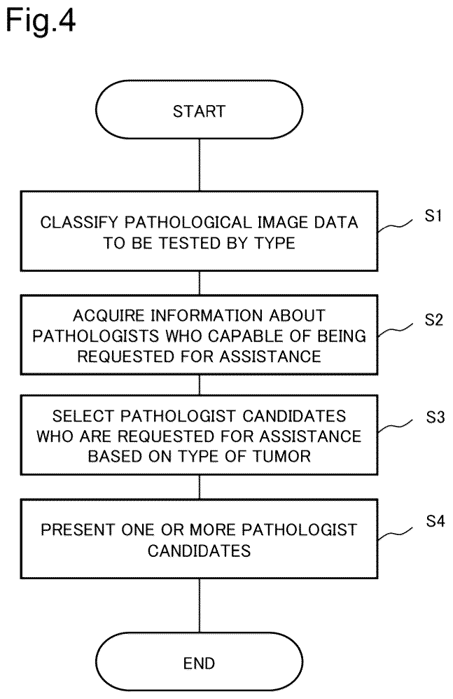

The first example embodiment will be described with reference to FIGS. 3 to 4.

### Inspection Assistance Device 10

With reference to FIG. 3, components included in the inspection assistance device 10 according to the first present example embodiment will be described. FIG. 3 is a block diagram illustrating a configuration of the inspection assistance device 10. As illustrated in FIG. 3, the inspection assistance device 10 includes a classification unit 12, a selection unit 13, and a presentation unit 14.

The classification unit 12 classifies the test target by tumor cell type using the pathological image data as the test target of the genetic test. The classification unit 12 is an example of a classification means.

In an example, the classification unit 12 uses a classifier that has been machine trained on characteristics of tumor cells appearing in a sample of pathological image data. The classifier is, for example, a convolutional neural network (CNN). Tag information indicating the correct answer of the type of tumor cell is added to each sample of the pathological image data in advance.

The classification unit 12 refers to the tag information added to the sample of the pathological image data and trains the classifier (CNN) in such a way that the type of tumor cell appearing in the sample of the pathological image data can be correctly identified. The classification unit 12 classifies the pathological image data as the test target by type using the classifier that has been trained in this manner, and then outputs information indicating the type of tumor cell related to the test target to the selection unit 13. For example, the information indicating the type of tumor cell related to the test target may be information indicating the likelihood for each tumor cell.

The selection unit 13 selects a pathologist candidate (hereinafter, it is simply referred to as a candidate) who is requested for assistance for a genetic test from among pathologists who are capable of being requested for assistance based on the type of tumor cell related to the test target. The selection unit 13 is an example of a selection means.

In an example, the selection unit 13 receives information indicating the type of tumor cell related to the test target from the classification unit 12. Next, the selection unit 13 refers to the database of pathologists in the server 300 (FIG. 1). The database of pathologists stores information and data about each pathologist together with identification information about each pathologist.

In the database of pathologists, the identification information about each pathologist is associated with information indicating the ability of each pathologist, such as the years of experience and specialized fields of each pathologist. Furthermore, in the database of pathologists, the identification information about each pathologist is also associated with a record of determination by tumor cell type. Alternatively/additionally, the identification information about each pathologist may be associated with the score of determination by tumor cell type. The score of determination by tumor cell type indicates how accurately the pathologist can determine the type of tumor cell. The score of determination by tumor cell type may be statistically calculated based on the record of the determination for each type of tumor cell, or may be manually given by the evaluator.

The selection unit 13 checks, for each pathologist registered in the database of pathologists, a record or a score of determination regarding the type of tumor cell related to the test target. Then, the selection unit 13 compares a record or a score of determination regarding the type of tumor cell related to the test target between all the pathologists registered in the database of pathologists.

For example, the selection unit 13 compares the number of times of determination regarding the type of tumor cell related to the test target between all the pathologists registered in the database of pathologists, and selects a predetermined number of pathologists as candidates in descending order of the number of times. Alternatively, the selection unit 13 may select a predetermined number of pathologists as candidates from among all the pathologists registered in the database of pathologists in descending order of the score of determination regarding the type of tumor cell related to the test target.

In a modification, the database of pathologists may be listed. In this case, each pathologist may be registered on the list according to his or her attribute, ability, expertise, experience, assessment, or load (workload).

The list of pathologists is a summary of pathologists with some common point. For example, the list includes pathologists working in a single hospital. Alternatively, the list includes pathologists belonging to any of the hospitals belonging to the same group. In yet other examples, the list includes pathologists who are in a particular region or a country.

In the above modification, the selection unit 13 also receives, from the terminal 200 of the pathologist, a request for assistance and information identifying any list registered in the database of pathologists. The selection unit 13 selects a candidate from the identified list.

In this manner, the selection unit 13 selects pathologist candidates who are requested for assistance for a genetic test. The selection unit 13 outputs information indicating the candidate selected from the database of pathologists to the presentation unit 14. The information indicating the candidate includes identification information about the candidate such as the name and affiliation of the candidate. Furthermore, the information indicating the candidate may include information indicating the ability of the candidate and information about a record or a score of determination regarding the type of tumor cell related to the test target.

The presentation unit 14 presents pathologist candidates. Presentation unit 14 is an example of a presentation means. The presentation here means information obtained through five senses. Specifically, the presentation unit 14 clearly indicates who is the candidate by any of characters, symbols, numbers, designs, sounds, and vibrations or a combination thereof.

In an example, presentation unit 14 receives information indicating a candidate from the selection unit 13. The presentation unit 14 creates a list of candidates to transmit the list of candidates (list data) to the terminal 200 of the pathologist (FIG. 1). Then, the presentation unit 14 controls the terminal 200 of the pathologist to display a list of candidates on the screen of the terminal 200 of the pathologist.

### Operation of Inspection Assistance Device 10

The operation of the inspection assistance device 10 according to the first present example embodiment will be described with reference to FIG. 4. FIG. 4 is a flowchart illustrating a flow of processing performed by each unit of the inspection assistance device 10.

As illustrated in FIG. 4, the classification unit 12 classifies the test target by tumor cell type using the pathological image data as the test target of the genetic test (S1). The classification unit 12 outputs information indicating the type of pathological image data to the selection unit 13.

Based on the information indicating the type of pathological image data, the selection unit 13 acquires information about the pathologists who are capable of being requested for assistance from a database (not illustrated) of pathologists stored in the server 300 (FIG. 1) (S2).

The selection unit 13 selects a candidate who is request for assistance for a genetic test from among pathologists who are capable of being requested for assistance based on the type of tumor cell (S3). The selection unit 13 outputs information indicating the candidate to the presentation unit 14.

The presentation unit 14 presents the candidate (S4). In an example, the presentation unit 14 displays the list of candidates on the screen of the terminal 200 (FIG. 1) of the pathologist.

Thus, the operation of the inspection assistance device 10 ends.

### Modifications

In a modification, at least some functions of the components of the inspection assistance device 10 may be implemented not in the server 300 but by a memory and a processor included in the terminal 200 of the pathologist. Specifically, all functions of the components of the inspection assistance device 10 may be implemented in the terminal 200 of the pathologist.

In the present modification, the inspection assistance device 10 performs the processing from steps S1 to S4 described above in the terminal 200 of the pathologist. In step S2, the selection unit 13 refers to the database of pathologists stored in the server 300. However, the selection unit 13 may refer to a copy of the database of pathologists recorded in advance in a storage unit (not illustrated) included in the terminal 200 of the pathologist. In this case, the inspection assistance device 10 does not need to refer to the server 300 in step S2. Therefore, the inspection assistance device 10 can perform the above-described operation even in an offline environment.

### Effects of Present Example Embodiment

According to the configuration of the present example embodiment, the classification unit 12 classifies the test target by tumor cell type using the pathological image data as the test target of the genetic test. The selection unit 13 selects pathologist candidates who are requested for assistance for a genetic test from among pathologists who are capable of being requested for assistance based on the type of tumor cell. The presentation unit 14 presents pathologist candidates. The user determines a pathologist who is requested for assistance from among the presented candidates. Then, the user determines whether to use the pathological image data for the genetic test while receiving assistance such as advice from the pathologist. As a result, it is possible to assist a genetic test using pathological image data.

### Second Example Embodiment

The second example embodiment will be described with reference to FIGS. 5 to 7. In the second present example embodiment, the same reference numerals are given to the components common to the first present example embodiment, and the description of the components will be omitted.

### System 2

An example of a configuration of a system 2 to which the inspection assistance device 20 according to the second present example embodiment is applied will be described with reference to FIG. 5. FIG. 5 is a diagram schematically illustrating an example of a configuration of the system 2.

As illustrated in FIG. 5, in addition to the terminal 200 of the pathologist, the system 2 further includes a cloud server 300 and terminals 400b, 400c, and 400d of other pathologists Dr. B, Dr. C, and Dr. D, respectively. The number of other pathologists is not particularly limited. All of other pathologists Dr. B, Dr. C, and Dr. D are assumed to be pathologists who are capable of being requested for assistance.

The terminal 200 of the pathologist (user) and the terminals 400b to 400d of other pathologists are connected to the common cloud server 300. The terminal 200 of the pathologist and the terminals 400b to 400d of other pathologists may refer to data on the cloud server 300. The terminal 200 of the pathologist and the terminals 400b to 400d of other pathologists can upload data onto the cloud server 300 or download data from the cloud server 300.

The inspection assistance device 20 according to the second present example embodiment is configured on the cloud server 300. The inspection assistance device 20 will be described below.

### Inspection Assistance Device 20

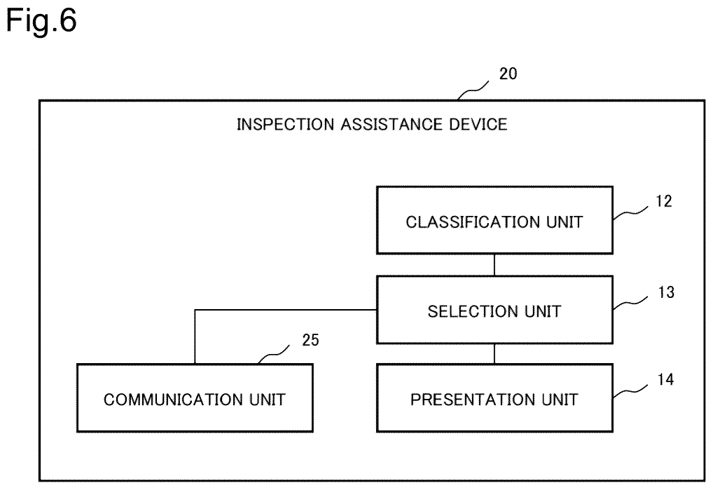

With reference to FIG. 6, components included in the inspection assistance device 20 according to the second present example embodiment will be described. FIG. 6 is a block diagram illustrating a configuration of the inspection assistance device 20. As illustrated in FIG. 6, the inspection assistance device 20 includes the classification unit 12, the selection unit 13, the selection unit 13, and the presentation unit 14. The inspection assistance device 20 further includes a communication unit 25.

The communication unit 25 transmits data related to the test target to an address that can be accessed by a pathologist (hereinafter, referred to as an observer) designated from among pathologist candidates who are requested for assistance. The communication unit 25 is an example of a communication means.

For example, it is assumed that the terminal 200 of the pathologist (user) designates another pathologist Dr. B as an observer. The communication unit 25 receives information indicating other pathologists Dr. B to Dr. D from the selection unit 13. The communication unit 25 receives information designating the observer from the terminal 200 of the pathologist. In an example, the observer is manually designated by the user of the terminal 200 of the pathologist. Alternatively, as in a modification described later, the inspection assistance device 20 may designate the observer.

The communication unit 25 transmits data related to the test target to the address of the cloud server 300 (FIG. 5) accessible by the terminal 400b of the designated observer. At this time, the communication unit 25 may restrict the right of access to the data related to the test target in such a way that the terminals 400c and 400d of other pathologists who are not observers cannot refer to or download the data related to the test target on the cloud server 300.

### Operation of Inspection Assistance Device 20

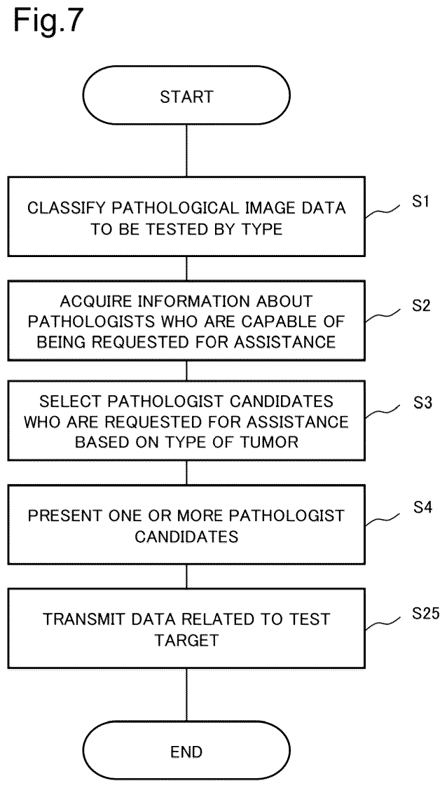

The operation of the inspection assistance device 20 according to the second present example embodiment will be described with reference to FIG. 7. FIG. 7 is a flowchart illustrating a flow of processing performed by each unit of the inspection assistance device 20.

As illustrated in FIG. 7, the classification unit 12 classifies the test target by tumor cell type using the pathological image data as the test target of the genetic test (S1). The classification unit 12 outputs information indicating the type of pathological image data to the selection unit 13.

Based on the information indicating the type of pathological image data, the selection unit 13 acquires information about the pathologists who are capable of being requested for assistance from a database (not illustrated) of pathologists stored in the server 300 (FIG. 1) (S2).

The selection unit 13 selects a candidate who is request for assistance for a genetic test from among pathologists who are capable of being requested for assistance based on the type of tumor cell (S3). The selection unit 13 outputs information indicating the candidates to the presentation unit 14 and the communication unit 25.

The presentation unit 14 presents the candidate (S4). In an example, the presentation unit 14 displays the list of candidates on the screen of the terminal 200 (FIG. 1) of the pathologist.

The communication unit 25 transmits data related to the test target to an address accessible by a pathologist (that is, observer) designated from among the candidates (S25).

Thus, the operation of the inspection assistance device 20 ends.

### Modifications

In a modification, at least some functions of the components of the inspection assistance device 20 may be implemented not in the cloud server 300 but by a memory and a processor included in the terminal 200 of the pathologist.

Specifically, all functions of the components of the inspection assistance device 20 may be implemented in the terminal 200 of the pathologist. In this case, the terminal 200 of the pathologist is communicably connected to the terminals 400b to 400d of other pathologists via an any network.

In the present modification, the inspection assistance device 20 performs the processing from steps S1 to S4 described above in the terminal 200 of the pathologist. In step S2, the selection unit 13 refers to the database of pathologists stored in the server 300. However, the selection unit 13 may refer to a copy of the database of pathologists recorded in advance in a storage unit (not illustrated) included in the terminal 200 of the pathologist. In this case, the inspection assistance device 20 does not need to refer to the server 300 in step S2.

Thereafter, the pathologist inputs information designating the observer to the terminal 200 of the pathologist. In step S25, the communication unit 25 transmits data related to the test target to the terminals 400b to 400d of other pathologists via an any network.

According to the configuration of the present modification, the inspection assistance device 20 can perform the above-described operation even in an offline environment.

In another modification, the inspection assistance device 20 designates an observer from among pathologists who are capable of being requested for assistance. Specifically, the inspection assistance device 20 refers to a record or a score of determination regarding the type of tumor cell related to the test target in the database of pathologists. Then, the inspection assistance device 20 scores a record or a score of determination regarding the type of tumor cell related to the test target between the pathologists who are capable of being requested for assistance. For example, the inspection assistance device 20 uses a calculation expression created in such a way that the larger the number of times of determination regarding the type of tumor cell related to the test target, the higher the score is. However, the scoring method is not limited thereto. The inspection assistance device 20 scores each of pathologists who are capable of being requested for assistance, and designates a certain number of pathologists as observers in descending order of scores.

### Effects of Present Example Embodiment

According to the configuration of the present example embodiment, the classification unit 12 classifies the test target by tumor cell type using the pathological image data as the test target of the genetic test. The selection unit 13 selects pathologist candidates who are requested for assistance for a genetic test based on the type of tumor cell. The presentation unit 14 presents pathologist candidates. The user determines a pathologist who is requested for assistance from among the presented candidates. Then, the user determines whether to use the pathological image data for the genetic test while receiving assistance such as advice from the pathologist. This can simplify the genetic test using pathological image data.

Furthermore, according to the configuration of the present example embodiment, the communication unit 25 that transmits data related to the test target to an address accessible by the pathologist designated from among the pathologist candidates who are requested for assistance is further included. As a result, the designated pathologist can access the data regarding the test target and perform determination related to the test target.

### Third Example Embodiment

The third example embodiment will be described with reference to FIGS. 8 to 10. In the third present example embodiment, the same reference numerals are given to constituent elements common to the first or second present example embodiment, and description of the constituent elements will be omitted.

### System 3

An example of a configuration of a system 3 to which the inspection assistance device 30 according to the third present example embodiment is applied will be described with reference to FIG. 8. FIG. 8 is a diagram schematically illustrating an example of a configuration of the system 3.

As illustrated in FIG. 8, in addition to the terminal 200 of the pathologist, the system 3 further includes a cloud server 300 and terminals 400b, 400c, and 400d of other pathologists Dr. B, Dr. C, and Dr. D. The number of other pathologists is not particularly limited. All of other pathologists Dr. B, Dr. C, and Dr. D are assumed to be pathologists who are capable of being requested for assistance.

The terminal 200 of the pathologist (user) and the terminals 400b to 400d of other pathologists are connected to the common cloud server 300. The terminal 200 of the pathologist and the terminals 400b to 400d of other pathologists may refer to data on the cloud server 300. The terminal 200 of the pathologist and the terminals 400b to 400d of other pathologists can upload data onto the cloud server 300 or download data from the cloud server 300.

On the cloud server 300, the inspection assistance device 30 according to the third present example embodiment is configured. The inspection assistance device 30 will be described below.

### Inspection Assistance Device 30

With reference to FIG. 9, components included in the inspection assistance device 30 according to the third present example embodiment will be described. FIG. 9 is a block diagram illustrating a configuration of the inspection assistance device 30. As illustrated in FIG. 9, the inspection assistance device 30 includes the classification unit 12, the selection unit 13, the selection unit 13, and the presentation unit 14. The inspection assistance device 20 further includes the communication unit 25 and an output unit 36.

The output unit 36 outputs information based on the determination result of the test target. The output unit 36 is an example of an output means.

In an example, the output unit 36 receives, from the communication unit 25, a determination result related to the test target by the observer. Then, the output unit 36 generates information based on the determination result of the test target to output the information. More specifically, the output unit 36 outputs information indicating how the observer has determined the type of tumor cell. For example, the information may explicitly identify the type of tumor cell, or may probabilistically evaluate the possibility that the type of tumor cell is any of A, B, . . . .

### Operation of Inspection Assistance Device 30

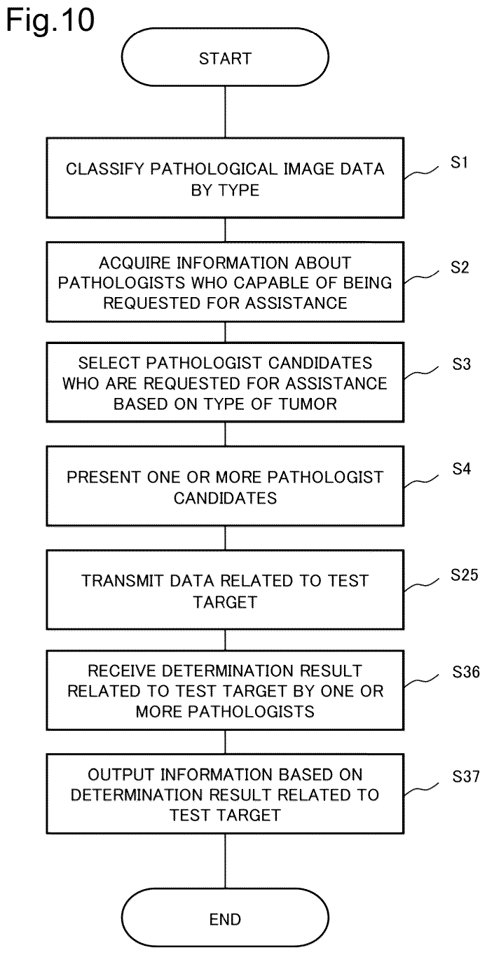

The operation of the inspection assistance device 30 according to the third present example embodiment will be described with reference to FIG. 10. FIG. 10 is a flowchart illustrating a flow of processing performed by each unit of the inspection assistance device 10.

As illustrated in FIG. 10, the classification unit 12 classifies the test target by tumor cell type using the pathological image data as the test target of the genetic test (S1). The classification unit 12 outputs information indicating the type of pathological image data to the selection unit 13.

Based on the information indicating the type of pathological image data, the selection unit 13 acquires information about pathologists who are capable of being requested for assistance from a database (not illustrated) stored in the server 300 (FIG. 1) (S2). The selection unit 13 outputs information about the pathologists who are capable of being requested for assistance to the selection unit 13.

The selection unit 13 selects pathologist candidates who are requested for assistance for a genetic test based on the type of tumor cell (S3). The selection unit 13 outputs, to the presentation unit 14, information indicating the pathologist candidates who are requested for assistance.

The presentation unit 14 presents pathologist candidates (hereinafter, the candidate may be simply referred to as candidates) who are requested for assistance (S4). In an example, the presentation unit 14 displays the list of candidates on the screen of the terminal 200 (FIG. 1) of the pathologist.

The user (pathologist) designates the pathologist as an observer from the list of candidates. The terminal 200 (FIG. 1) of the pathologist uploads information indicating the pathologist designated as the observer by the user to the cloud server 300 (FIG. 8) accessible by the observer terminal. It is assumed that the user designates another pathologist Dr. B as an observer.

The communication unit 25 transmits data related to the test target to the terminal 400b (FIG. 8) of another pathologist (Dr. B) designated as the observer by the user (S25).

The observer inputs the determination result related to the test target to the terminal 400b (FIG. 8) of another pathologist. The terminals 400b to 400d (FIG. 8) of other pathologists upload the determination result regarding the test target by the observer to the cloud server 300.

The communication unit 25 receives the determination result related to the test target by the observer from the terminal 400b (FIG. 8) of another pathologist (S36). The communication unit 25 outputs a determination result on a test target by the observer to the output unit 36.

The output unit 36 outputs information based on the determination result of the test target by the observer to the terminal 200 of the pathologist (S37).

Thus, the operation of the inspection assistance device 30 ends.

### Modifications

In a modification, at least some functions of the components of the inspection assistance device 30 may be implemented not in the cloud server 300 but by a memory and a processor included in the terminal 200 of the pathologist.

Specifically, all functions of the components of the inspection assistance device 30 may be implemented in the terminal 200 of the pathologist. In this case, the terminal 200 of the pathologist is communicably connected to the terminals 400b to 400d of other pathologists via an any network.

In the present modification, the inspection assistance device 30 performs the processing from steps S1 to S4 described above in the terminal 200 of the pathologist. Thereafter, the pathologist inputs information designating the observer to the terminal 200 of the pathologist. In step S25, the communication unit 25 transmits data related to the test target to the terminal 400b of another pathologist Dr. B designated as an observer via an any network.

In step S36, the communication unit 25 receives the determination result of the test target by the observer from the terminals 400b to 400d of other pathologists via an any network. In step S37, the output unit 36 outputs information based on the determination result of the test target by the observer to a display device included in the terminal 200 of the pathologist or an external device connected to the terminal 200 of the pathologist.

According to the configuration of the present modification, the inspection assistance device 30 can perform the above-described operation even in an offline environment.

In another modification, the inspection assistance device 20 may score pathologists who are capable of being requested for assistance based on the type of tumor cell, and designate a certain number of pathologists as observers in descending order of score. The description of a modification of the second example embodiment is cited, and the detailed description of the present modification is omitted.

### Effects of Present Example Embodiment

According to the configuration of the present example embodiment, the classification unit 12 classifies the test target by tumor cell type using the pathological image data as the test target of the genetic test. The selection unit 13 selects pathologist candidates who are requested for assistance for a genetic test based on the type of tumor cell. The presentation unit 14 presents pathologist candidates. The user determines a pathologist who is requested for assistance from among the presented candidates. Then, the user determines whether to use the pathological image data for the genetic test while receiving assistance such as advice from the pathologist. As a result, it is possible to assist a genetic test using pathological image data.

Furthermore, according to the configuration of the present example embodiment, the communication unit 25 transmits data related to the test target to an address accessible by the pathologist designated from among the pathologist candidates who are requested for assistance. As a result, the designated pathologist can access the data regarding the test target and perform determination related to the test target.

According to the configuration of the present example embodiment, the output unit 36 outputs information based on the determination result of the test target. The user determines whether to use the pathological image data for a genetic test while referring to the information based on the determination result of the test target. This can simplify the genetic test using pathological image data.

### Hardware Configuration

Each component of the inspection assistance devices 10 to 30 described in the first to third example embodiments represents a functional unit block. Some or all of these components are implemented by an information processing device 900 as illustrated in FIG. 8, for example. FIG. 8 is a block diagram illustrating an example of a hardware configuration of the information processing device 900.

As illustrated in FIG. 8, the information processing device 900 includes the following configuration as an example.
central processing unit (CPU) 901
read only memory (ROM) 902
random access memory (RAM) 903
program 904 loaded into RAM 903
storage device 905 storing program 904
drive device 907 that reads and writes recording medium 906
communication interface 908 connected to a communication network 909
input/output interface 910 for inputting/outputting data
bus 911 connecting respective components

Each component of the inspection assistance devices 10 to 30 described in the first to third example embodiments is achieved by the CPU 901 reading and executing the program 904 for achieving these functions. The program 904 for achieving the function of each component is stored in the storage device 905 or the ROM 902 in advance, for example, and the CPU 901 loads the program into the RAM 903 and executes the program as necessary. The program 904 may be supplied to the CPU 901 via the communication network 909, or may be stored in advance in the recording medium 906, and the drive device 907 may read the program and supply the program to the CPU 901.

According to the above configuration, the inspection assistance devices 10 to 30 described in the first to third example embodiments are achieved as hardware. Therefore, effects similar to the effects described in the first to third example embodiments can be obtained.

Although the present invention is described with reference to the example embodiments (and examples), the present invention is not limited to the above example embodiments (and examples). Various modifications that can be understood by those of ordinary skill can be made to the configurations and details of the above example embodiments (and examples) within the scope of the present invention.

Some or all of the above example embodiments may be described as the following Supplementary Notes, but are not limited to the following.

### Supplementary Note

### Supplementary Note 1

An inspection assistance device including
a classification means configured to classify, using pathological image data as a test target of a genetic test, the test target by tumor cell type,
a selection means configured to select one or more pathologist candidates who are requested for assistance for the genetic test from among pathologists who are capable of being requested for assistance based on a type of tumor cell related to the test target, and
a presentation means configured to present the pathologist candidate.

### Supplementary Note 2

The inspection assistance device according to Supplementary Note 1, wherein
the selection means selects one or more pathologist candidates who are requested for assistance for the genetic test based on the type of tumor cell related to the test target from a list of pathologists having a common point.

### Supplementary Note 3

The inspection assistance device according to Supplementary Note 1 or 2, further including
a communication means configured to transmit data related to the test target to an address accessible by a pathologist designated from among the pathologist candidates who are requested for assistance.

### Supplementary Note 4

The inspection assistance device according to Supplementary Note 3, wherein
the communication means transmits data related to the test target together with an analysis result of the test target using a computer to an address accessible by the pathologist.

### Supplementary Note 5

The inspection assistance device according to Supplementary Note 4, wherein
the communication means uploads data related to the test target to a cloud server accessible by the pathologist.

### Supplementary Note 6

The inspection assistance device according to any one of Supplementary Notes 3 to 5, wherein
the communication means receives a determination result of the test target by the pathologist, and wherein
the inspection assistance device further comprises an output means configured to output information based on the determination result of the test target.

### Supplementary Note 7

The inspection assistance device according to Supplementary Note 6, wherein
the output means outputs information based on the determination result of the test target together with pathological image data as the test target.

### Supplementary Note 8

The inspection assistance device according to Supplementary Note 6 or 7, wherein
the output means statistically analyzes the determination result of the test target by the pathologist to output information based on the analysis result.

### Supplementary Note 9

An inspection assistance method including
classifying, using pathological image data as a test target of a genetic test, the test target by tumor cell type,
selecting one or more pathologist candidates who are requested for assistance for the genetic test from among pathologists who are capable of being requested for assistance from among pathologists who are capable of being requested for assistance based on a type of tumor cell related to the test target, and
presenting the pathologist candidates.

### Supplementary Note 10

A non-transitory recording medium storing a program for causing a computer to execute
classifying, using pathological image data as a test target of a genetic test, the test target by tumor cell type,
selecting one or more pathologist candidates who are requested for assistance for the genetic test from among pathologists who are capable of being requested for assistance based on a type of tumor cell related to the test target, and
presenting the pathologist candidates.

### Supplementary Note 11

The inspection assistance device according to Supplementary Note 2, wherein
the list includes one or more pathologists belonging to a same hospital.

### Supplementary Note 12

The inspection assistance device according to Supplementary Note 2, wherein
the list includes one or more pathologists belonging to a same group of hospitals.

### Supplementary Note 13

The inspection assistance device according to Supplementary Note 2, wherein
the list includes one or more pathologists belonging to a particular region or a particular country.

### REFERENCE SIGNS LIST

10 inspection assistance device
12 classification unit
13 selection unit
14 presentation unit
20 inspection assistance device
25 communication unit
30 inspection assistance device
36 output unit

What is claimed is:

1. An inspection assistance device comprising:
a memory configured to store instructions; and
at least one processor configured to execute the instructions to perform:
classifying, using pathological image data as a test target of a genetic test, the test target by tumor cell type;
selecting one or more pathologist candidates who are requested for assistance for the genetic test from among pathologists who are capable of being requested for assistance based on a type of tumor cell related to the test target; and
presenting the pathologist candidates.

2. The inspection assistance device according to claim 1, wherein
the at least one processor is configured to execute the instructions to perform:
selecting one or more pathologist candidates who are requested for assistance for the genetic test from a list of pathologists having a common point.

3. The inspection assistance device according to claim 1, wherein
the at least one processor is configured to execute the instructions to perform:
transmitting data related to the test target to an address accessible by a pathologist designated from among the pathologist candidates who are requested for assistance.

4. The inspection assistance device according to claim 3, wherein
the at least one processor is configured to execute the instructions to perform:
transmitting data related to the test target together with an analysis result of the test target using a computer to an address accessible by the pathologist.

5. The inspection assistance device according to claim 4, wherein
the at least one processor is configured to execute the instructions to perform:
uploading data related to the test target to a cloud server accessible by the pathologist.

6. The inspection assistance device according to claim 3, wherein
the at least one processor is configured to execute the instructions to perform:
receiving a determination result of the test target by the pathologist, and
outputting information based on the determination result of the test target.

7. The inspection assistance device according to claim 6, wherein
the at least one processor is configured to execute the instructions to perform:
outputting information based on the determination result of the test target together with pathological image data as the test target.

8. The inspection assistance device according to claim 6, wherein
the at least one processor is configured to execute the instructions to perform:
statistically analyzing the determination result of the test target by the pathologist to output information based on the analysis result.

9. An inspection assistance method comprising:
classifying, using pathological image data as a test target of a genetic test, the test target by tumor cell type;
selecting one or more pathologist candidates who are requested for assistance for the genetic test from among pathologists who are capable of being requested for assistance based on a type of tumor cell related to the test target; and
presenting the pathologist candidates.

10. A non-transitory recording medium storing a program for causing a computer to execute:
classifying, using pathological image data as a test target of a genetic test, the test target by tumor cell type;
selecting one or more pathologist candidates who are requested for assistance for the genetic test from among pathologists who are capable of being requested for assistance based on a type of tumor cell related to the test target; and
presenting the pathologist candidates.

11. The inspection assistance device according to claim 2, wherein
the list includes one or more pathologists belonging to a same hospital.

12. The inspection assistance device according to claim 2, wherein
the list includes one or more pathologists belonging to a same group of hospitals.

13. The inspection assistance device according to claim 2, wherein
the list includes one or more pathologists belonging to a particular region or a particular country.
